(19) United States
(12) Patent Application Publication
(10) Pub. No.: US 2014/0134631 A1
(43) Pub. Date: May 15, 2014

(54) CENTRIFUGAL MICROFLUIDIC PLATFORM

(71) Applicants: Liviu Clime, Longueuil (CA); Maxence Mounier, Boucherville (CA); Emmanuel Roy, Montreal (CA); Teodor Veres, Montreal (CA)

(72) Inventors: Liviu Clime, Longueuil (CA); Maxence Mounier, Boucherville (CA); Emmanuel Roy, Montreal (CA); Teodor Veres, Montreal (CA)

(73) Assignee: NATIONAL RESEARCH COUNCIL OF CANADA, Ottawa, ON (CA)

(21) Appl. No.: 14/128,079

(22) PCT Filed: Mar. 22, 2012
(86) PCT No.: PCT/CA2012/000262

§ 371 Date: Dec. 20, 2013

Related U.S. Application Data
(60) Provisional application No. 61/504,273, filed on Jul. 4, 2011.

(51) Int. Cl. C12Q 1/68

(52) U.S. Cl. CPC C12Q 1/686
USPC 435/6.12; 435/287.2

(57) ABSTRACT

A centrifugal microfluidic device is provided having a microfluidic circuit, a fluid reservoir for providing fluid in the microfluidic circuit, a hydrodynamic resistance element in fluid communication with the reservoir for controlling rate of flow of a fluid out of the reservoir, and a siphoned chamber in fluid communication with the hydrodynamic resistance element and the microfluidic circuit for receiving fluid from the hydrodynamic resistance element and for delaying and metering of the fluid into the microfluidic circuit. The microfluidic device is useful for performing a biological assay. Operation of the device is completely independent on the liquid-solid contact angle and wetting properties of the liquids on the solid material of the platform, and the device does not need a carefully controlled rotation protocol.

### CROSS-REFERENCE TO RELATED APPLICATIONS

This application claims the benefit of U.S. Provisional Patent Application Ser. No. 61/504,273 filed Jul. 4, 2011, the entire contents of which is herein incorporated by reference.

### FIELD OF THE INVENTION

The present invention is related to microfluidic devices and their use in performing biological assays.

### BACKGROUND OF THE INVENTION

Centrifugal (rotating) microfluidic devices have been gaining in importance since they can provide precise control and manipulation of very small amounts of liquids, for example a few microliters only (Jia 2004; Zoval 2004; Madou 2006; Ducree 2007). Recently, several applications of centrifugal microfluidic platforms like staining assays (Chen 2010), whole-cell sensing (Date 2010), real-time PCR (Jia 2004; Focke 2010) and single-molecule detection (Melin 2005) have been demonstrated. In actual (traditional) devices, temporal and spatial control of liquids are achieved by controlling in-plane structure of the microfluidic channels as well as the wetting properties of the materials used for fabricating the chips (Zoval 2004; Lu 2006; Ducree 2007). The most important microfluidic elements based on these ideas are the capillary valves (Madou 2006; Ducree 2007) and the siphon valves (Siegrist 2009) and they are designed to stop and release the liquids at different places or at different rotation frequencies of the disk, respectively. By programming an appropriate protocol, several microfluidic functions can in principle be achieved by using these elements. There are however several difficulties with these elements and as for the capillary valves the most important are:
The burst frequencies of capillaries depends hyperbolically on their distance from the rotation center. Consequently, in regions near the rotation center, the errors in the spatial positioning of these valves become larger.
Capillary valves are effective for a very narrow range of liquid-solid contact angles (Lu 2006; Ducree 2007). Consequently, not all liquids can be manipulated on a disk made of a given material.
Since a capillary valve opens at a specific rotation frequency, all of the subsequent microfluidic circuit related to a certain valve must be designed for that frequency. This can be a serious drawback if different liquids, released at different frequencies by different capillary valves, have to flow through the same region of a microfluidic circuit.

On the other hand, siphon valves consist of radial siphons near a reservoir, whose crests are designed above the liquid level in these reservoirs (Siegrist 2009). The liquid remains in this siphon while the disk rotates but it slowly advances by capillary action toward the crest as the disk stops. Once the liquid has primed the crest, the disk can rotate again and flush the liquid from the reservoir into the microfluidic circuit. The main concerns with this approach are:
The siphon channel walls have to be hydrophilic with respect to the liquid, otherwise no capillary rise toward the siphon crest is possible (Siegrist 2009). This is a serious limitation for most applications since very few materials used in fabrication of microfluidic devices meet such criteria for aqueous solutions.
Often, microbiological protocols require the control of more than two liquids on the same microfluidic platform. A typical waiting queue for the liquids released at the end of the protocol consists of a multiple siphon structure whose number of crests equals the number of protocol steps skipped by the corresponding liquid. As the number of crests to overcome increases, the necessary time for the liquid to prime a crest increases, needlessly lengthening the biological protocol.
As the number of loops in the valve increases, the necessary footprint gets larger accordingly (Siegrist 2009). This is a serious difficulty since siphon valves usually operate close to the disk center next to the liquid reservoirs, where a large amount of the footprint is already occupied by these reservoirs.
Since the priming of siphon valves is based on capillary rise dynamics, any imperfection or defect in the fabrication process can act as a stop for the liquid meniscus and the liquid remains trapped in the reservoir.

By the nature of microfluidic devices, almost all design features in microfluidic devices are based on some combination of channels and chambers, resulting in considerable apparent similarity in the features of one device compared to another. However, many of these apparently similar features are actually quite different since they perform different functions in the device. The function of a feature depends on its relationship to other features in the device and where in the device the feature exists. Thus, the configuration and interconnection of features is of great importance in understanding the actual purpose and nature of the features described in any particular document.

U.S. Pat. No. 6,143,248 in Example 2 and FIG. 12 describes a centrifugal microfluidic device that employs several parallel and identical channels connecting two reservoirs. The siphon of reservoir E is a venting channel rather than a fluid siphon. Moreover, the liquid is never supposed to reach this siphon since this would annihilate all capillary valves at the entrance of reservoir E. Metering is achieved by capillary filling all channels B with liquid and overflowing the rest of the liquid through channel C. This is be done by controlling the rotation speed in such a way that surplus liquid from reservoir E flows through channel C to the waste while metered liquid is kept in place in the array of channels by the capillary valves at the end of these channels. By increasing the rotation speed, liquid from channels B is transferred to reservoir E (this is in fact the metered liquid). Consequently, the metered liquid in this case is given by the total volume of the array of capillaries B. The limitations of this principle based on capillary valves are addressed above.

U.S. Pat. No. 7,497,996 describes a centrifugal microfluidic device for transferring fluids between multiple reservoirs by making use of sudden variations in the angular velocity of the disc (angular accelerations) and related circumferential inertial forces. The channel 8A is not a siphon and it is not designed for metering either. It has a curved shape similar to that of a siphon but the actual shape is designed according to the angular velocity protocol used to actuate the platform. There is no crest point on channel 8A. Further, this device is extremely sensitive to the rotation protocol as the timing has to be almost perfect in order to be able to move the liquids.

United States Patent Publication 2003-0152491 describes an application of centrifugal pneumatic pumping where pneumatic energy stored in a compression chamber is used to push liquids back to the rotation center. By using this pumping technique, back and forth movements of two or more liquids in a serpentine channel are used to enhance diffusive mixing between species contained in these liquids. The serpentine channel described in this document does not provide hydrodynamic resistance. Paragraph [0037] states that its volume must be many times larger than the reservoir itself in order to be able to accommodate the whole volume of liquid from the reservoir. Thus, this channel is a huge reservoir rather than that a serpentine channel. Further, accurate control of rotation speed and rotation protocol are mandatory and the reservoirs described in this document are “compression chambers” that do not perform any function of metering or delay.

United States Patent Publication 2005-0136545 is directed to mixing of different fluids on a centrifugal microfluidic platform in order to perform specific reactions and assays. Paragraph [0064] states that the flow is controlled by tuning cross-sectional area of the channels, not channel length. Paragraph [0076] as well as [0090] indicate that the length of the serpentine channel is in the millimeter range and that this channel is a mixing chamber rather than a hydrodynamic resistance for delaying fluids on CD.

In United States Patent Publication 2002-0151078, as described in paragraphs [0102] and [0103], serpentine channels are used to enhance mixing between multiple species and not for providing hydrodynamic resistance.

U.S. Pat. No. 7,152,616 describes centrifugal pneumatic pumping where liquids in a microfluidic device are actuated by using the suction force created by a buffer liquid in an air chamber (602). Although some feature shapes are similar to a siphon channel, the underlying principle does not rely on the geometry of these channels. There is no metering, no delay and rotation protocol is still needed.

Thus, there remains a need for centrifugal microfluidic platforms that overcome one or more of the problems associated with existing platforms.

### SUMMARY OF THE INVENTION

There is provided a centrifugal microfluidic device comprising: a microfluidic circuit; a fluid reservoir for providing fluid in the microfluidic circuit; a hydrodynamic resistance element in fluid communication with the reservoir for controlling rate of flow of a fluid out of the reservoir; and, a siphoned chamber in fluid communication with the hydrodynamic resistance element and the microfluidic circuit for receiving fluid from the hydrodynamic resistance element and for delaying and metering of the fluid into the microfluidic circuit.

There is further provided a use of a microfluidic device of the present invention for performing a biological assay.

There is further provided a method of performing a biological assay using a microfluidic device of the present invention.

A microfluidic device of the present invention is generally monolithic having microfluidic structures engineered into a solid substrate. The solid substrate may comprise any suitable material. It is an advantage of the present invention that the nature of the material is unimportant to the operation of the device. Some examples of materials suitable for the solid substrate include a polymer (e.g. a thermoplastic, a thermoset, an elastomer), silicon, glass or mixtures thereof. The solid substrate preferably comprises a polymer, more preferably a thermoplastic elastomer (TPE) or polydimethylsiloxane (PDMS). The device comprises a microfluidic circuit having at least one micro-scale channel in fluid communication with at least one microfluidic chamber. Channels include, for example, sample loading channels, cell loading channels, medium perfusion channels, mixing channels, particle separation or fractionation channels, gradient generating channels and high resistance perfusion conduits, which may have different channel dimensions dictated by the specific application. Microfluidic chambers include, for example, cell culture chambers, bacteria or cell capture chambers, biomolecular interaction chambers or mixing chambers. Other microfluidic structures may also be present, for example valves and pumps for controlling fluid flow, conduits, inlets, outlets, and the like, but it is a special advantage of the present invention that such valves and pumps may be unnecessary for the operation of the device. Channels are preferably no larger than 1 mm, at least in one direction, and the total length of the device is preferably on the order of a few centimeters to tens of centimeters. The depth of chambers, including the reservoir and siphoned chamber, may be larger than the depth of the channels in order to accommodate larger volumes of fluid, and may exceed 1 mm in size. Microfluidic devices can be readily fabricated by any of the actual microfabrication techniques known in the art, for example, machining, hot embossing, 3D printing, etc.

The fluid reservoir for providing fluid in the microfluidic circuit may be a chamber present in the microfluidic device. The reservoir should be able to hold sufficient fluid to successfully perform whatever is being required of the microfluidic circuit. The reservoir has an outlet through which fluid can flow into the hydrodynamic resistance element.

The hydrodynamic resistance element controls the rate of fluid flow out of the fluid reservoir into the siphoned chamber. The hydrodynamic resistance element provides a hydrodynamic resistance to impede the flow of fluid without actually stopping the flow. A properly designed hydrodynamic resistance element can replace a valve structure for controlling fluid flow, thereby mitigating against the problems of wettability and high pressure associated with the use of valves. The hydrodynamic resistance element has a geometry that is designed to provide the desired hydrodynamic resistance. The hydrodynamic resistance element should be designed with a view to the amount of time (Δt) that the fluid is to be queued in the siphoned chamber before being delivered into the microfluidic circuit. Such design involves various parameters including: the volume of fluid to be released to the microfluidic circuit and which may be initially added to the reservoir; the positions of the fluid level, reservoir outlet, siphoned chamber inlet and siphon crest point with respect to rotation center of the device; the cross-sectional area of the reservoir; the cross-sectional area of the hydrodynamic resistance element; the density of the liquid; and, the angular velocity of the microfluidic platform. Preferably, the hydrodynamic resistance element is designed to satisfy the relationship expressed in Eq. 1a:

$$Δ t = \frac{2 V [ \frac{{R}_{2} - {R}_{1}}{{G}_{1} {w}_{1} {h}_{1}} + \frac{{L}_{h}}{{G}_{2} {w}_{2} {h}_{2}} ]}{p {ω}^{2} [ {R}_{3}^{2} - {R}_{1}^{2} ]} \qquad ( Eq . 1 a )$$

where V is the volume of fluid to be released into the microfluidic circuit, Lₕ is the total length of the hydrodynamic resistance element, R₁ is the position of the fluid level with respect to the rotation center of the device, R₂ is the position of the reservoir outlet with respect to the rotation center of the device, R₃ is the position of the inlet to the siphoned chamber with respect to the rotation center of the device, w₁ and h₁ are the dimensions of the cross-sectional area of the reservoir, w₂ and h₂ are the dimensions of the cross-sectional area of the hydrodynamic resistance element, p is the density of the fluid and ω is the angular velocity (radians/second) of the microfluidic device. The quantities G₁ and G₂ are related to the cross-sectional areas of the reservoir and the hydrodynamic resistance element through the generalized formula expressed in Eq. 1 b:

$$\begin{matrix}{G}_{i} = \frac{64}{{π}^{4} η} {\sum }_{m = 1}^{∞} {\sum }_{n = 1}^{∞} \frac{1}{{( 2 m - 1 )}^{2} {( 2 n - 1 )}^{2} {λ}_{mn}} \\ where & ( Eq . 1 b ) \\ {λ}_{mn} = \frac{{β}_{m}^{2}}{{w}_{i}^{2}} + \frac{{β}_{n}^{2}}{{h}_{i}^{2}} ; {β}_{m} = π ( m - \frac{1}{2} ) ; {β}_{n} = π ( n - \frac{1}{2} ) & ( Eq . 1 c )\end{matrix}$$

The fluid is released into the microfluidic circuit when fluid level in the siphoned chamber reaches the crest point of the siphon. At this point, the exact amount of fluid released into the microfluidic circuit is given by Eq. 2:

V=w_{C}h_{C}ΔR_{C} (Eq. 2)

where V is defined above, w_{C} is the width of the chamber of the siphoned chamber, h_{C} is the height of the chamber of the siphoned chamber, R_{c} is the position of the siphon crest point with respect to the rotation center of the device, and ΔR_{C} is the radial distance between the crest point of the siphon and the point where the chamber and siphon of the siphoned chamber meet.

Thus, the time (Δt) needed for releasing a volume of fluid (V) into the microfluidic circuit from the siphoned chamber can be obtained by combining Eq. 1a with Eq. 2 to give Eq. 3:

$$Δ t = \frac{2 {w}_{c} {h}_{c} Δ {R}_{c} [ \frac{{R}_{2} - {R}_{1}}{{G}_{1} {w}_{1} {h}_{1}} + \frac{{L}_{h}}{{G}_{2} {w}_{2} {h}_{2}} ]}{p {ω}^{2} [ {R}_{3}^{2} - {R}_{1}^{2} ]} \qquad ( Eq . 3 )$$

It is evident from Eq. 3 that the queue time in the siphoned chamber (Δt) is proportional to the length (Lₕ) of the hydrodynamic resistance element. Since the queue time in the siphoned chamber is related to the timing of delivery of the fluid to the microfluidic circuit, such timing can be readily controlled by controlling the length of the hydrodynamic resistance element, for a fixed cross-sectional area of the hydrodynamic resistance element. The hydrodynamic resistance element may have any shape and structure that provides a hydrodynamic resistance. A shape and structure obeying the relationship in Eq. 3 is suitable. The device can accommodate any shape between inlet and outlet in order to obtain the desired hydrodynamic resistance, however, the hydrodynamic resistance element preferably comprises a channel, for example a serpentine channel.

It should be noted that while serpentine channels and other curved channels are known in the art, none are designed to provide a hydrodynamic resistance. The intended use of such channels in the prior art is different and the shapes of such channels arise in applications where a longer path for diffusive mixing of species is desired.

The hydrodynamic resistance element used for controlling the flow rate presents several advantages over simple straight and rectangular microfluidic constrictions. First, due to several limitations in microfabrication techniques, it is relatively difficult to precisely control the cross-sectional area of the constriction, mainly along the Z-axis (deepness). For a fixed length of channel there will always exist a value of the cross-sectional area of the channel in order to develop a desired flow rate. However, when the flow rates are too small this is not always possible since several biological samples contain particles and aggregates that can agglomerate and clog the channel. In the present invention, the hydrodynamic resistance is controlled by channel length instead of the cross-sectional area. This parameter is easier to control since for a given thickness, appropriate lengths and widths can be designed in order to accommodate different flow rates. Second, the topological features of the channel minimize the effect of the driven pressure on the developed flow since the pressure difference between the ends of one single element in the channel is zero. This is due to the fact that each element is circumferentially oriented with respect to the rotation center. In this way, the hydrodynamic resistance can be substantially increased while the driven centrifugal pressure hardly changes from one end of the channel to the other.

The siphoned chamber comprises a metering and delay chamber coupled at an outlet to a siphon channel. The metering and delay chamber receives fluid at an inlet from the hydrodynamic resistance element at a specific rate thereby filling the metering and delay chamber at a specific rate. The siphon channel comprises at least two branches pointing in the direction of centrifugal force and oriented radially (i.e. in the rotational plane of the device). The two branches are connected by a U-turn where the bottom of the U is a crest of the siphon channel. Control of the distance between the inlet of the metering and delay chamber and the crest of the siphon channel together with control of the length of the hydrodynamic resistance element leads to control over the timing of fluid delivery into the microfluidic circuit, without the need for valves and independent of material and rotation protocols of the device. Thus, the fluid reservoir, hydrodynamic resistance element, metering and delay chamber and siphon are configured with respect to the center of rotation of the device so that the metering and delay chamber is filled and then emptied all at once in a pre-determined time interval to deliver fluid to the microfluidic circuit.

Traditional use of siphon channels in the prior art has been for delaying liquids at the outlet of reservoirs and releasing the liquids into the microfluidic circuit after a full stop of the microfluidic platform when the liquid advances by capillary action toward the crest and eventually overcomes it, allowing the microfluidic protocol to continue. The difficulty with this traditional approach is that the siphon channel has to be hydrophilic with respect to the liquid condition and this is a difficult condition to fulfill for the majority of materials used in microfluidics. Often, complicated and time consuming treatments to tune up the wetting properties of these materials have been necessary. In the present invention, the wetting properties of the microfluidic channel plays no role since the crest is overcome naturally by filling the metering and delay chamber since the position of the crest along this chamber is designed accordingly. Once the fluid in the metering and delay chamber attains a critical level corresponding to priming of the crest, the fluid is flushed suddenly into the microfluidic circuit. The flow rate for this flushing can be properly adjusted by tailoring the geometry of the siphon's output channel.

Thus, the present invention provides a design for centrifugal microfluidic devices, which comprises coupling a hydrodynamic resistance element to a siphoned chamber for delay and metering in order to properly delay fluids in microfluidic protocols. The combination of the hydrodynamic resistance element and the siphoned chamber form a temporization and metering circuit that can be used in microfluidic devices to control flow of fluid into the microfluidic circuit of the device. The rate of the flow from the fluid reservoir at its outlet is controlled by the hydrodynamic resistance. Since the hydrodynamic resistance element drains directly into the metering and delay chamber, the filling time of this chamber is also well controlled by the geometry of the hydrodynamic resistance element. As the level of the fluid in the metering and delay chamber reaches the siphon crest, the fluid is drained entirely through the siphon channel into the microfluidic circuit.

Advantageously, the platform of the present invention can accommodate any liquid, can be fabricated in any material and does not need any rotation protocol. The operation of the device is completely independent on the liquid-solid contact angle and wetting properties of the liquids on the solid material of the platform. Thus, surface tension coefficients become irrelevant and time-consuming surface treatments of the device for improving wetting properties are unnecessary. In addition, the device occupies a much smaller lateral footprint than a multiple siphon valve and the same device can be used for several different applications without design adjustments. Further, since the platform rotates at constant speed, several liquids flowing to a common region of the microfluidic circuit will behave in the same manner. Furthermore, it is possible to control the time intervals at which different liquids are introduced into the microfluidic circuit while spinning the platform at a constant rotation frequency. In fact, absolute control over rotational frequency of the platform is not required and no rotation protocol is required since metering of liquids into the microfluidic circuit is not completely dependent of rotation frequency and is completely independent of any rotation protocol that may be used. Thus, the platform does not require valves and electronic components to operate valves, and does not even require electricity since the motor can be mechanically driven. This is a tremendous advantage for medical diagnostics, especially in third world countries where access to electricity is problematic. Finally, devices of the present invention have longer shelf-lives than prior art devices of similar nature, which is particularly advantageous for diagnostic applications.

Microfluidic devices of the present invention are useful for performing biological assays including use as miniaturized reactors, fluidic systems, cell culture platforms, microfluidic biosensors, sample preparation platforms and other devices for performing biological assays.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts a schematic diagram of a temporization and metering circuit in accordance with the present invention using a hydrodynamic resistance element and a siphoned chamber, including geometrical parameters related to its design in the context of a centrifugal microfluidic device;

FIG. 2A depicts a schematic diagram of a valveless microfluidic circuit comprising two fluid reservoirs linked to a waste reservoir through two hydrodynamic resistance elements of different lengths coupled to two siphoned chambers of the same size;

FIG. 2B depicts a graph showing chamber filling time dependence on the number of loops in hydrodynamic resistance elements of different lengths, where the bullets are experimentally determined points and the line is a theoretical prediction;

FIG. 3A depicts a schematic diagram of proposed elements in a complex Lysis&PCR microfluidic setup utilizing a temporization and metering circuit in accordance with the present invention;

FIG. 3B depicts a schematic diagram of proposed elements in a microfluidic hybridization application utilizing a temporization and metering circuit in accordance with the present invention; and,

FIG. 3C depicts a schematic diagram of an implementation of the proposed elements in FIGS. 3A and 3B in a complete microfluidic device on a compact disc (CD).

### DESCRIPTION OF PREFERRED EMBODIMENTS

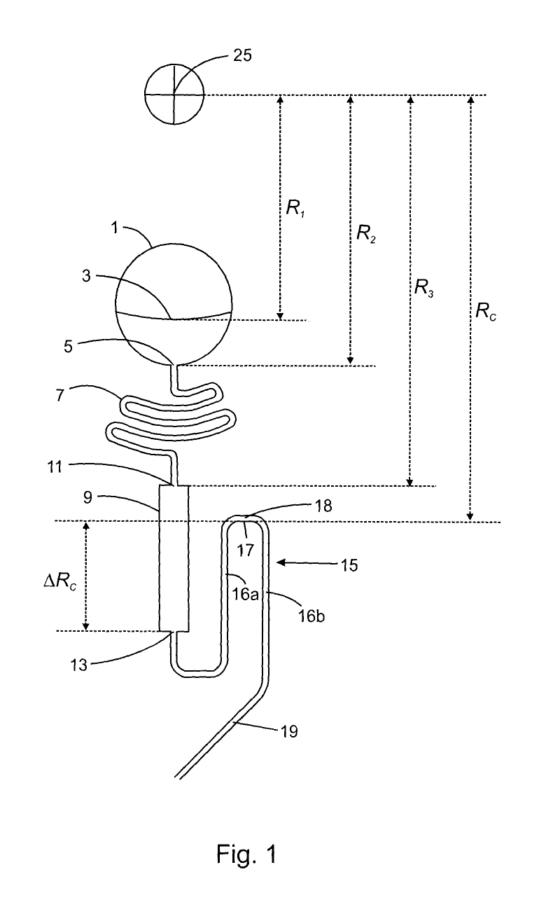

Referring to FIG. 1, a temporization and metering circuit in accordance with the present invention is depicted that may be used in a centrifugal microfluidic device. The temporization and metering circuit comprises a hydrodynamic resistance element in the form of serpentine channel 7 in fluid communication via chamber inlet 11 with a siphoned chamber in the form of metering and delay chamber 9 in fluid communication via chamber outlet 13 with siphon 15. The rate of fluid flow from fluid reservoir 1 at reservoir outlet 5 is controlled by the hydrodynamic resistance provided by serpentine channel 7. Since the serpentine channel drains directly into metering and delay chamber 9, the filling time of chamber 9 is also well controlled by the geometry of the serpentine channel. As the level of the fluid in chamber 9 reaches siphon crest 17, the fluid is drained entirely from chamber 9 through siphon 15 into a microfluidic circuit (not shown) in fluid communication with siphon outlet channel 19. Siphon 15 is in the plane of rotation of the device and the siphon comprises U-turn 18 connecting two arms 16a,16b pointing in the direction of centrifugal force (towards rotation center 25) where arm 16a feeds fluid into the U-turn and arm 16b carries fluid away from the U-turn. Crest point 17 is located at the most distal point in U-turn 18.

For properly timed filling and draining of metering and delay chamber 9, the elements of the temporization and metering circuit are configured with respect to rotation center 25 of the device in accordance with Eq. 1 above. R₁ is the radial distance between rotation center 25 and initial fluid level 3 in fluid reservoir 1. R₂ is the radial distance between rotation center 25 and reservoir outlet 5. R₃ is the radial distance between rotation center 25 and metering and delay chamber inlet 11. R_{C} is the radial distance between rotation center 25 and crest point 17 in U-turn 18 of siphon 15. ΔR_{C} is the radial distance between crest point 17 and metering and delay chamber outlet 13. For fixed values of R₁, R₂, R₃, R_{C}, initial fluid volume in fluid reservoir 1, cross-sectional dimensions of reservoir 1 and serpentine channel 7, fluid density and angular velocity of the device, the time interval for fluid delivery from the temporization and metering circuit into the microfluidic circuit is readily controlled by controlling only one parameter—the length of serpentine channel 7.

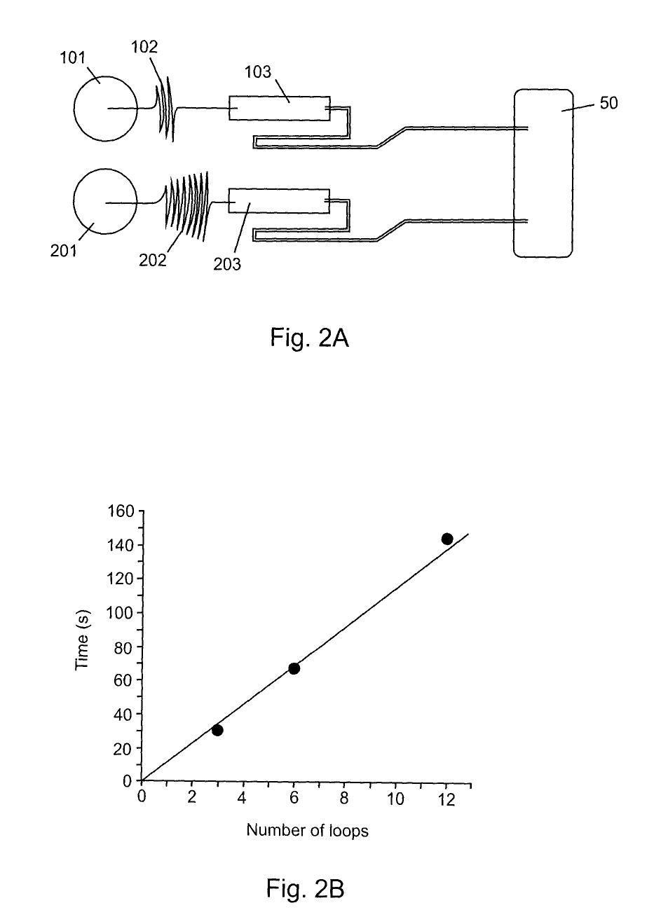

Referring to FIG. 2A, a simple valveless microfluidic circuit comprising two fluid reservoirs 101,201 linked to waste reservoir 50 through two hydrodynamic resistance elements 102,202 of different lengths coupled to two siphoned chambers 103,203 of the same size is depicted to illustrate the use of a temporization and metering circuit in a simple device. The system of these two coupled elements is able to perform several microfluidic functions. For example, for temporization of fluids in reservoirs, fluids can be queued into the siphoned chambers and released in the microfluidic circuit at very precise time intervals. The two valveless microfluidic circuits shown in FIG. 2A have identical siphoned chambers for metering and delay but different temporization serpentines (different number of loops) for providing hydrodynamic resistance to fluid flow from the fluid reservoirs. The measured delay between the moment when the platform begins to rotate and the release of the liquid from the chamber to the waste reservoir are shown as bullets in FIG. 2B. As shown in FIG. 2B, there is a good agreement between experiment and the expected behavior demonstrating the efficacy of the present invention to precisely control fluid flow by adjusting in-plane design features instead of out-of-plane (Z) design features. Further, since the release of fluids from the siphoned chamber can be controlled by the position of the siphon crest, precise metering of the released amount of fluid can be achieved by positioning the crest of the siphon along the metering and delay chamber.

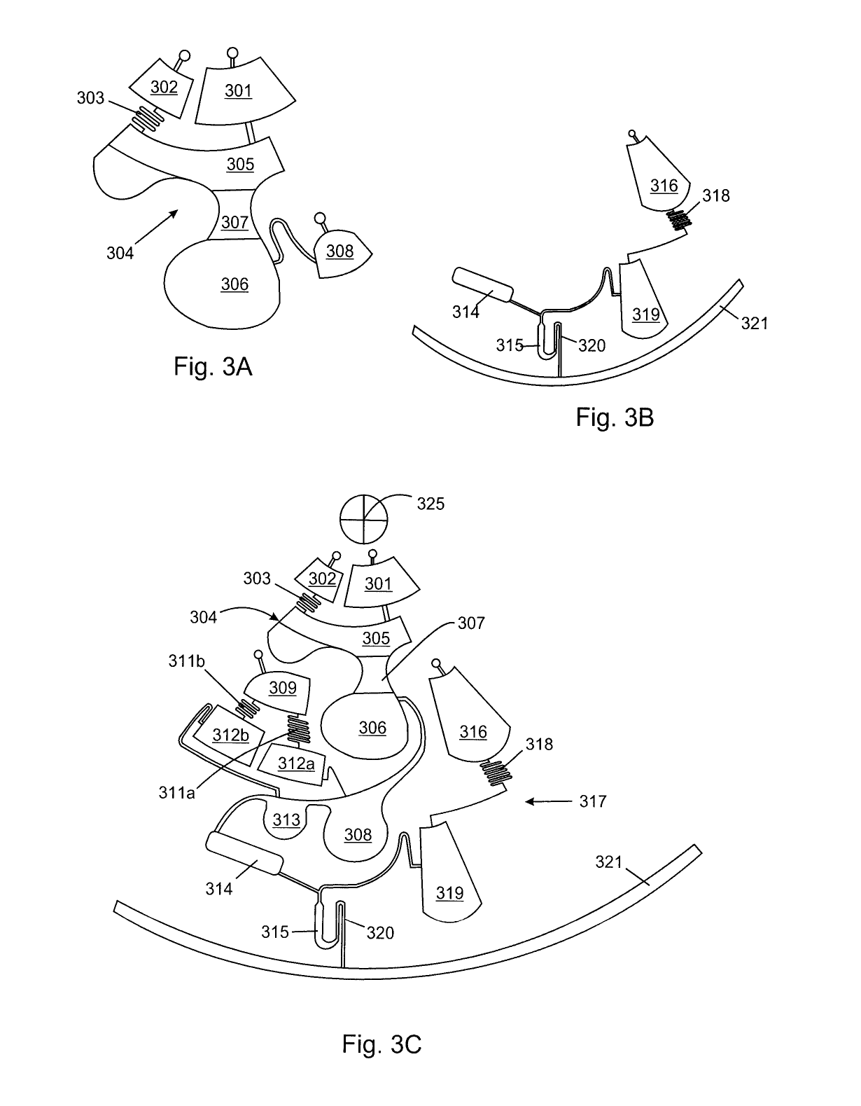

FIG. 3 depicts application of temporization and metering circuits in accordance with the present invention to a proposed microfluidic device for conducting actual biological assays. Among the numerous operations that can be integrated on a CD using the present invention, lysing of cells, metering for precise dilution of solutes and DNA hybridization are illustrated in FIG. 3. FIG. 3A depicts proposed elements in a complex microfluidic circuit for sample lysis followed by PCR amplification of the lysed sample. FIG. 3B depicts proposed elements in a microfluidic hybridization application. And, FIG. 3C depicts implementation of the proposed elements in FIGS. 3A and 3B in a complete microfluidic device on a compact disc (CD), where a hybridization experiment is conducted on the PCR amplified sample.

Referring to FIG. 3, the lysis/PCR circuit comprises sample reservoir 301 and PCR buffer reservoir 302 in fluid communication with lysis, metering and mixing chamber 304. Flow of PCR buffer into chamber 304 is controlled by serpentine loop 303. Sample and PCR buffer are mixed in mixing area 305 before reaching lysing area 306 where the sample is lysed by grinding on plastic beads. Capillary valve 307 controls flow from mixing area 305 to lysing area 306. Lysing area 306 is in fluid communication with PCR reaction chamber 308 where PCR is performed on lysed samples. PCR reaction chamber 308 also receives a metered flow of oil from oil reservoir 309, the flow of oil being temporized and metered by a temporization and metering circuit that comprises serpentine channel 311a providing a hydrodynamic resistance and siphoned chamber 312a for metering and delaying oil flow to the PCT reaction chamber 308. PCR reaction chamber 308 is in fluid communication with exo chamber 313 where PCR products prepared in PCR reaction chamber 308 are mixed with Klenow enzyme. Exo chamber 313 also receives a flow of oil from oil reservoir 309, the flow of oil being temporized and metered by a temporization and metering circuit that comprises serpentine channel 311b providing a hydrodynamic resistance and siphoned chamber 312b for metering and delaying oil flow to the exo chamber 313. Exo chamber 313 is in fluid communication with resuspension chamber 314 for resuspending PCR products before entering hybridization chamber 315 for conducting the hybridization experiment. Hybridization chamber 315 is supplied with a flow of washing buffer from washing buffer reservoir 316, the flow of washing buffer being temporized an metered by temporization and metering circuit 317 that comprises serpentine channel 318 providing a hydrodynamic resistance and siphoned chamber 319 for metering and delaying flow of the washing buffer to hybridization chamber 315. Retention loop 320 retards the flow of fluid out of the hybridization chamber before the fluid reaches waste reservoir 321. As illustrated in FIG. 3C, the temporization and metering circuits all lie in the plane of rotation of the device and are all configured with respect to rotation center 325 to provide the temporization and metering function as described in the present invention.

In addition to the advantages already discussed above, there are additional advantageous aspects to the device illustrated in FIG. 3. In the PCR sample preparation procedure, operations can be simplified by including sequential PCR buffer transfer. The sample (after lysis) and the buffer reservoirs are delayed with respect to each other by using temporization and metering circuits of the present invention. The transfer of the solution to the PCR chamber can be realized by using the same principle. Further, a current issue in hybridization experiments is to accurately control the proper filling of the hybridization chamber. In prior art centrifugal microfluidic this is difficult to achieve because of the many physical parameters to control simultaneously (angular velocity, position on the disc, inflow and outflow liquid rates, wetting properties, etc.). By using the present approach, proper filling of the hybridization chamber is ensured regardless the rotation speed, the position of the chamber on the disc or the flow rates at the inlet and outlet of the chamber.

### REFERENCES

The contents of the entirety of each of which are incorporated by this reference.
Andersson P, Ekstrand G, et al. (2008) Structural Units that Define Fluidic Functions. U.S. Pat. No. 7,429,354 issued Sep. 30, 2008.
Chen H, Li X, et al. (2010) A rotating microfluidic array chip for staining assays. Talanta. 81(4-5), 1203-8.
Date A, Pasini P, et al. (2010) Integration of spore-based genetically engineered whole-cell sensing systems into portable centrifugal microfluidic platforms. Analytical and Bioanalytical Chemistry. 398, 349-356.
Ducree J, Haeberle S, et al. (2007) The centrifugal microfluidic Bio-Disk platform. Journal of Micromechanics and Microengineering. 17(7), S103-S115.
Focke M, Stumpf F, et al. (2010) Microstructuring of polymer films for sensitive genotyping by real-time PCR on a centrifugal microfluidic platform. Lab on a Chip. 10(19), 2519-26.
Gallagher S, Druyor-Sanchez R, et al. (2003) System and Methods for Mixing within a Microfluidic Device. United States Patent Publication 2003-0064507 published Apr. 3, 2003.
Haeberle S, Brenner T, Zengerleab R, Ducree J. (2006) Centrifugal extraction of plasma from whole blood on a rotating disk. Lab on a Chip. 6, 776-781.
Jia G, Ma K S, et al. (2004) CD (compact disc)-Based DNA hybridization and detection. Proceedings of SPIE—The International Society for Optical Engineering. Strasbourg.
Kellogg G, Kieffer-Higgins S G, Mian A. (2000) Capillary Microvalve. U.S. Pat. No. 6,143,248 issued Nov. 7, 2000.
Kellogg G J, Duffy D, Pelta A. (2002) Microfluidics Devices and Methods for High Throughput Screening. United States Patent Publication 2002-0151078 published Oct. 17, 2002.
Kellogg G J, Carvalho B L. (2003a) Bidirectional Flow Centrifugal Microfluidic Devices. United States Patent Publication 2003-0152491 published Aug. 14, 2003.
Kellogg G, Kieffer-Higgins S G, et al. (2003b) Devices and Methods for Using Centripetal Acceleration to Drive Fluid Movement in a Microfluidics System. U.S. Pat. No. 6,548,788 issued Apr. 15, 2003.
Lu C, Juang Y J, et al. (2006) Superhydrophobic valve for microfluidics. 64^{th} Annual Technical Conference—ANTEC, Conference Proceedings. Society of Plastics Engineers, Charlotte, N.C. pp 2566-2570.
Madou M, Zoval J, et al. (2006). Lab on a CD. Annual Review of Biomedical Engineering. 8, 601-628.
Mehta T B, Kopf-Sill A R, et al. (2003) Manipulation of Microparticles in Microfluidic Systems. U.S. Pat. No. 6,632,655 issued Oct. 14, 2003.
Melin J, Johansson H, et al. (2005) Thermoplastic microfluidic platform for single-molecule detection, cell culture, and actuation. Analytical Chemistry. 77(22), 7122-30.
Mian A, Kieffer-Higgins S G, Corey G D. (2004) Devices and Methods for Using Centripetal Acceleration to Drive Fluid Movement in a Microfluidics System. U.S. Pat. No. 6,709,869 issued Mar. 23, 2004.
Ozaki N, Oka H, et al. (2009) Liquid Delivery Apparatus and Liquid Delivery Method. U.S. Pat. No. 7,497,996 issued Mar. 3, 2009.
Peng X Y, Li P C H, Yu H-Z, Parameswaran M, Chou W L. (2007) Spiral microchannels on a CD for DNA hybridizations. Sensors and Actuators B. 128, 64-69.
Schmid N, Bansal P, Kellogg G. (2005) Microfluidics Devices and Methods for Performing Based Assays. United States Patent Publication 2005-0136545 published Jun. 23, 2005.
Siegrist J, Gorkin R, et al. (2009) Serial siphon valving for centrifugal microfluidic platforms. Microfluidics and Nanofluidics. 9, 55-63.
Valencia R M, Chen Y F, et al. (2003) Optical Bio-discs and Fluidic Circuits for Analysis of Cells and Methods Relating Thereto. United States Patent Publication 2003-0219713 published Nov. 27, 2003.
Weibel D B, Lee A, Potenta S, Siegel A, Kruithoff M, Whitesides G M. (2010) Valves and Reservoirs for Microfluidic Systems. United States Patent Publication 2010-0116343 published May 13, 2010.
Zoval J V, Madou M J. (2004) Centrifuge-based fluidic platforms. Proceedings of the IEEE. 92(1), 140-153.
Zucchelli P, Van de Vyver B. (2006) Devices and Methods for Programmable Microscale Manipulation of Fluids. U.S. Pat. No. 7,152,616 issued Dec. 26, 2006.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A centrifugal microfluidic device comprising: a microfluidic circuit; a fluid reservoir for providing fluid in the microfluidic circuit; a hydrodynamic resistance element in fluid communication with the reservoir for controlling rate of flow of a fluid out of the reservoir; and, a siphoned chamber in fluid communication with the hydrodynamic resistance element and the microfluidic circuit for receiving fluid from the hydrodynamic resistance element and for delaying and metering of the fluid into the microfluidic circuit.

2. The device according to claim 1 having a center of rotation, wherein the siphoned chamber comprises a metering and delay chamber and a siphon having a crest point.

3. The device according to claim 2, wherein the fluid reservoir, hydrodynamic resistance element, metering and delay chamber and siphon are configured with respect to the center of rotation so that the metering and delay chamber is filled and then emptied all at once in a pre-determined time interval to deliver fluid to the microfluidic circuit.

4. The device according to claim 2 having a plane of rotation, wherein rotation of the device produces a centrifugal force in a radial direction in the plane of rotation, and wherein the siphon comprises two branches oriented radially and pointing in the direction of centrifugal force.

5. The device according to claim 4, wherein the branches of the siphon are connected by a U-turn having a bottom defining the crest of the siphon.

6. The device according to claim 5, wherein the hydrodynamic resistance element and siphoned chamber are designed to satisfy Eq. 3:
$$Δ t = \frac{2 {w}_{c} {h}_{c} Δ {R}_{c} [ \frac{{R}_{2} - {R}_{1}}{{G}_{1} {w}_{1} {h}_{1}} + \frac{{L}_{h}}{{G}_{2} {w}_{2} {h}_{2}} ]}{p {ω}^{2} [ {R}_{3}^{2} - {R}_{1}^{2} ]} \qquad ( Eq . 3 )$$
where
Lₕ is total length of the hydrodynamic resistance element, R₁ is position of fluid level with respect to the center of rotation, R₂ is the position of the reservoir outlet with respect to the center of rotation, R₃ is the position of the inlet to the siphoned chamber with respect to the center of rotation, w₁ and h₁ are width and height of the cross-sectional area of the fluid reservoir, w₂ and h₂ are width and height of the cross-sectional area of the hydrodynamic resistance element, p is density of the fluid, ω is angular velocity in radians/second of the microfluidic device, w_{C} is width of the metering and delay chamber, h_{C} is height of the metering and delay chamber, ΔR_{C} is radial distance between the crest point of the siphon and an outlet of the metering and delay chamber to the siphon, G₁ and G₂ are related to the cross-sectional areas of the fluid reservoir and the hydrodynamic resistance element through generalized formula Eq. 1b:
$$\begin{matrix}{G}_{i} = \frac{64}{{π}^{4} η} {\sum }_{m = 1}^{∞} {\sum }_{n = 1}^{∞} \frac{1}{{( 2 m - 1 )}^{2} {( 2 n - 1 )}^{2} {λ}_{mn}} \\ where & ( Eq . 1 b ) \\ {λ}_{mn} = \frac{{β}_{m}^{2}}{{w}_{i}^{2}} + \frac{{β}_{n}^{2}}{{h}_{i}^{2}} ; {β}_{m} = π ( m - \frac{1}{2} ) ; {β}_{n} = π ( n - \frac{1}{2} ) . & ( Eq . 1 c )\end{matrix}$$

7. The device according to claim 5, wherein time that a fluid is queued in the siphoned chamber before being delivered into the microfluidic circuit is proportional to length of the hydrodynamic resistance element.

8. The device according to claim 1, wherein the hydrodynamic resistance element comprises a channel.

9. The device according to claim 8, wherein the channel is serpentine.

10. The device according to claim 1 further comprising a solid substrate within which the microfluidic circuit, fluid reservoir, hydrodynamic resistance element and siphoned chamber are engineered, and wherein control of fluid flow in the device is independent of wetting properties of the fluid on the substrate.

11. The device according to claim 10, wherein the solid substrate comprises a polymer, silicon, glass or a mixture thereof.

12. The device according to claim 10, wherein the solid substrate comprises a thermoplastic, a thermoset or an elastomeric polymer.

13. The device according to claim 10, wherein the solid substrate comprises a thermoplastic elastomer (TPE) or polydimethylsiloxane (PDMS).

14. The device according to claim 1, wherein control of fluid flow in the device is independent of rotation protocol of the device.

15. The device according to claim 1 for use in a method of performing a biological assay.

16. Use of a microfluidic device as defined in claim 1 for performing a biological assay.

17. Use of a microfluidic device as defined in claim 1 as a miniaturized reactor, a fluidic system, a cell culture platform, a microfluidic biosensor or a sample preparation platform.
